(19) United States
(12) Plant Patent Application Publication
(10) Pub. No.: US 2026/0090486 P1
(43) Pub. Date: Mar. 26, 2026

(54) Fragaria Plant Named 'Finette'

(50) ;

(71) Applicant: Flevo Berry Holding BV, ENS (NL)

(72) Inventor: Marcel Suiker, ENS (NL)

(73) Assignee: Flevo Berry Holding BV, ENS (NL)

(21) Appl. No.: 19/320,627

(22) Filed: Sep. 5, 2025

(30) Foreign Application Priority Data
Sep. 23, 2024 (QZ) 20242217

(51) Int. Cl. A01H 6/74; A01H 5/08

(52) U.S. Cl. CPC A01H 6/7409; A01H 5/08
USPC PLT/208

(57) ABSTRACT

A new and distinct Fragaria x ananassa plant named ‘Finette’ which exhibits an upright growth habit with moderately dense foliage and a relative abundance of stolons. ‘Finette’ is characterized by large foliage born on broadly outreaching stolons, an abundance of red berries with achenes that are level with the firm flesh which has a pleasantly sweet, non-acidic flavor proven to be consistently produced from late April to early November while maintaining their large conical size. ‘Finette’ has shown stability of these characteristics from generation to generation.

Latin name of the genus and species:

The Latin name of the genus and species of the novel variety disclosed herein is Fragaria x ananassa.

Variety denomination:

The inventive cultivar of Fragaria disclosed herein has been given the variety denomination ‘Finette’.

### BACKGROUND OF THE INVENTION

Parentage: ‘Finette’ is a seedling selection that resulted from a controlled pollination breeding program carried out in 2017 in Ens, Netherlands. The new variety originated as a seedling from the crossing of Fragaria x ananassa ‘13-077’ (not patented), the seed parent, with Fragaria x ananassa ‘07-075’ (not patented) as the pollen parent. Seed from said cross was harvested, then germinated, and the resulting seedlings were grown to a mature size in order to evaluate for desirable commercial characteristics. The claimed plant was then subjected to a series of trials from 2017 through 2023 to evaluate the distinctness and stability of the characteristics first observed. In 2024, the claimed plant was finally selected as a new and distinctive cultivar.

Asexual Reproduction: Asexual reproduction of ‘Finette’, by way of rooting stolon cuttings, was first performed in September of 2017 in Ens, Netherlands. Through several subsequent generations, the unique features of this cultivar have proven to be stable and true to type.

### SUMMARY OF THE INVENTION

The cultivar ‘Finette’ has not been observed under all possible environmental conditions, thus being so the phenotype may vary somewhat with variations in environment such as temperature, light intensity, and water availability, with no variance in genotype. The following traits have been repeatedly observed and are determined to be the unique characteristics of ‘Finette’. These characteristics in combination distinguish ‘Finette’ as a new and distinct Fragaria cultivar:
1. Fragaria ‘Finette’ exhibits an upright growth habit with moderately dense foliage and a relative abundance of stolons; and
2. Fragaria ‘Finette’ exhibits large foliage born on broadly outreaching stolons; and
3. Fragaria ‘Finette’ exhibits an abundance of berries produced from late April to early November maintaining size and consistent productivity; and
4. Fragaria ‘Finette’ exhibits large conical, red berries with achenes that are level with the firm flesh; and
5. Fragaria ‘Finette’ exhibits a pleasant yet non-acidic flavor.

### BRIEF DESCRIPTION OF THE FIGURES

FIG. 1 illustrates, as nearly true as it is reasonably possible to make the same in color photographs of this type, an exemplary 12-month-old ‘Finette’ plant, grown at a commercial greenhouse in Ens, Netherlands in a 19 cm container.

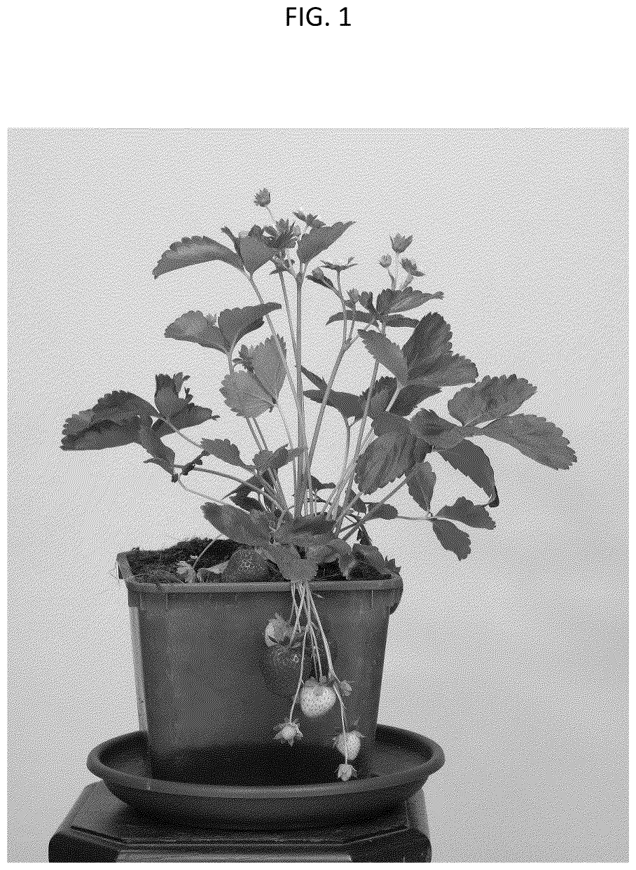

FIG. 2 illustrates, as nearly true as it is reasonably possible to make the same in color photographs of this type, the typical foliage of ‘Finette’.

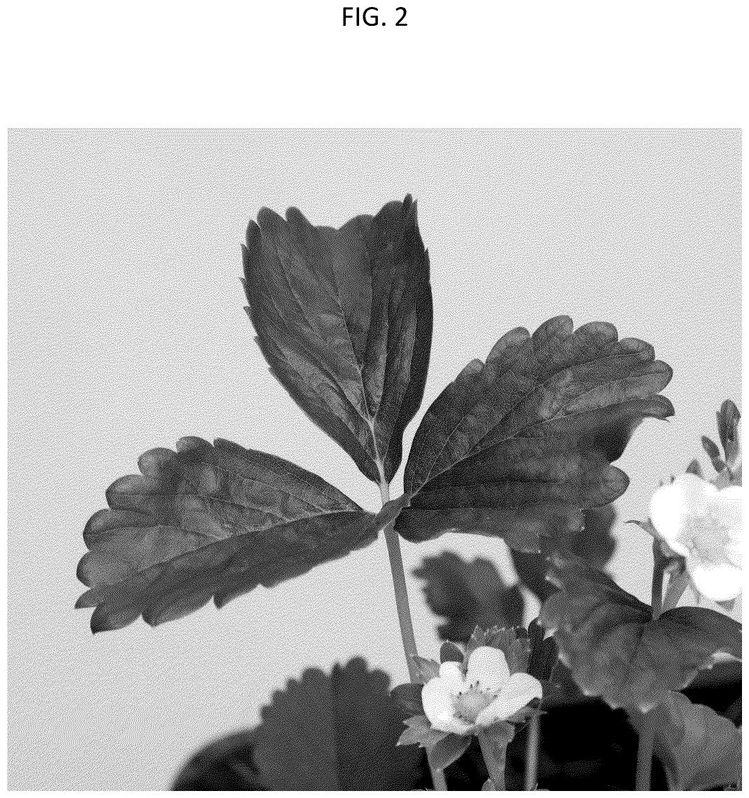

FIG. 3 illustrates, as nearly true as it is reasonably possible to make the same in color photographs of this type, the typical flower of ‘Finette’.

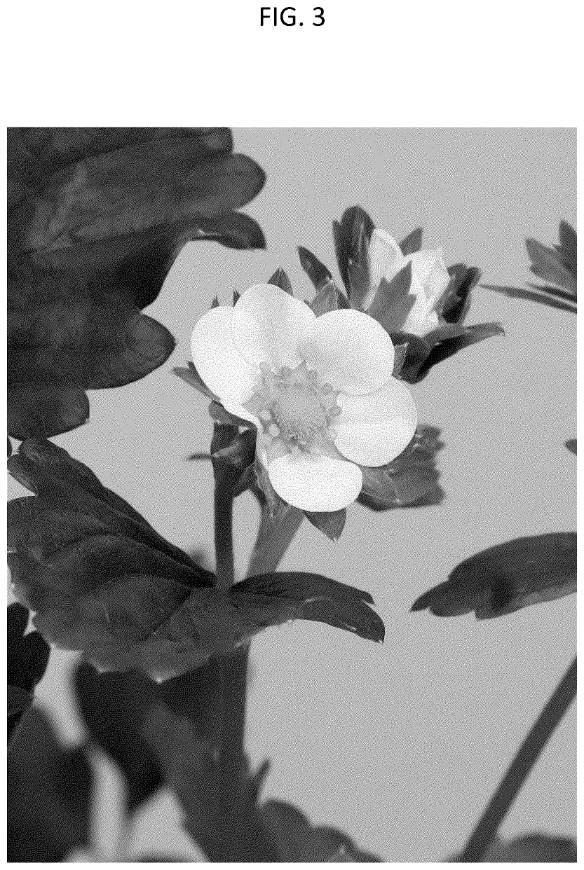

FIG. 4 illustrates, as nearly true as it is reasonably possible to make the same in color photographs of this type, the typical fruit of ‘Finette’.

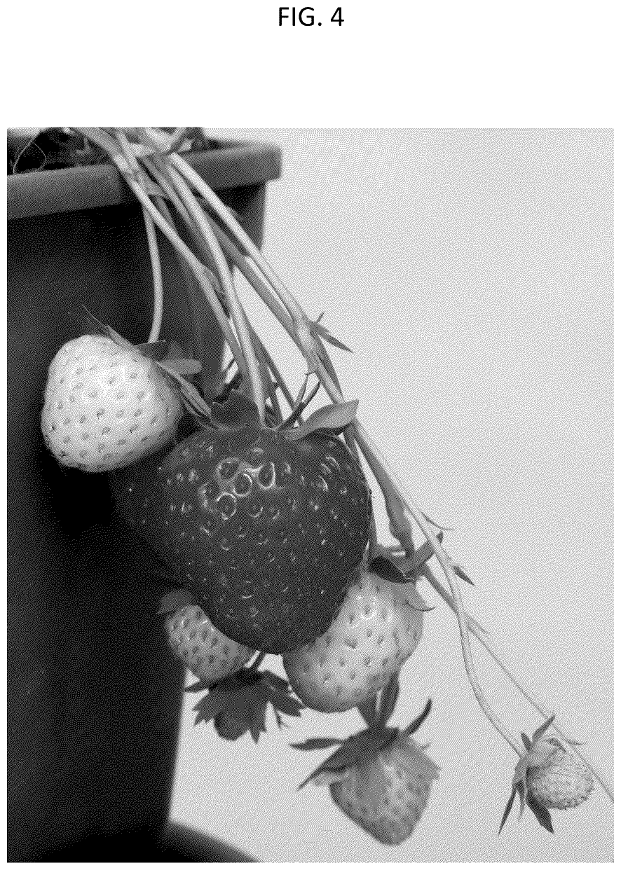

### BOTANICAL DESCRIPTION OF THE PLANT

The following is a detailed botanical description of a new and distinct variety of Fragaria known as ‘Finette’, based upon observations of 12-month-old greenhouse-grown plants in 19 cm nursery containers, produced in Ens, Netherlands. Plants were grown on benches in a non-climate-controlled greenhouse, using conventional production protocols for Fragaria plants which consisted of drip irrigation with fertigation applications as needed. Biological insect control measures were utilized in production. Plants were produced with full sun exposure and no photoperiodic treatments or artificial light was given to the plants. Temperatures ranged from 15 to 25 degrees Celsius during the day and 10 to 18 degrees at night. Observation data was recorded in September of 2024.

Those skilled in the art will appreciate that certain characteristics will vary with older or, conversely, with younger plants. ‘Finette’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climatic and cultural conditions. Color notations are based on The Royal Horticultural Society Colour Chart, The Royal Horticultural Society, London, 2019, reprint.

A botanical description of ‘Finette’ and a comparison with the parent plants and the most similar commercial Fragaria cultivar known to the inventor, is provided below.
Plant description:
   Growth habit.—Acaulescent, stoloniferous perennial; with leaves and inflorescence developing from basal rosettes with approximately 6 rosettes per pot; no lateral branching is present.
   Plant profile.—Flattened globular.
   Average height.—26.0 cm to the top of the foliar plane; 27.0 cm to the top of the floral plane.
   Average width.—43.9 cm.
   Propagation.—Mode of propagation — Asexual propagation is accomplished by rooting stolon cuttings.
Root system:
   Type.—Moderately fibrous root system, with adventitious rooting at stolon nodes.
   Branching.—Very freely branched.
   Density.—Moderately dense.
   Distribution.—The majority of the root system is somewhat shallow.
   Diameter.—Largest roots measuring 1.0 mm in diameter.
   Texture.—Smooth; lacking root hairs.
   Color.—White, RHS 155B.
Stolons:
   Attitude.—Pendulous.
   Cross-section.—Rounded.
   Length.—19.0 cm.
   Diameter.—0.25 cm.
   Internode length.—9.5 cm.
   Strength.—Strong.
   Texture.—Sparsely covered with short hairs; average length of hairs is 0.1 cm; colored translucent white, nearest to nearest to RHS NN155D.
   Luster.—Matte.
   Color.—Juvenile stolons, adaxial and abaxial surfaces — Yellow-green, nearest to RHS 144B. Mature stolons, adaxial and abaxial surfaces — Yellow-green, nearest to 144A. Internodes, adaxial and abaxial surfaces — Yellow-green, nearest to 144A.
Foliage:
   Arrangement.—Alternate in basal rosettes.
   Attachment.—Petiolate.
   Attitude.—Upward and outward.
   Division.—Trifoliate.
   Compound leaf outline.—Reniform.
   Length.—11.6 cm, excluding the petiole.
   Width.—12.9 cm.
   Quantity.—4 leaves per rosette.
   Leaflets.—Quantity — 3. Shape — Varying from broad ovate to broad obovate. Length — 7.1 cm. Width — 5.4 cm. Cross-section — Slightly concave. Apex — Very abruptly acute. Base — Broadly attenuate to near obtuse. Margin — Coarsely dentate to crenate. Texture, adaxial surface — Smooth and glabrous except for the veins which are moderately covered with very thin, soft adpressed pubescence with an average length of 0.5 mm; hairs are a translucent white, nearest to nearest to white, nearest to RHS NN155D. Texture, abaxial surface — Slightly rugose and glabrous except for the veins which are moderately covered with very thin, soft adpressed pubescence with an average length of 0.75 mm; hairs are a translucent white, nearest to nearest to white, nearest to RHS NN155D. Luster, adaxial surface — Glossy. Luster, abaxial surface — Matte. Color — Juvenile foliage color, adaxial surface — Green, nearest to RHS 143B. Juvenile foliage color, abaxial surface — Green, nearest to a blend of RHS 138B and 138C. Mature leaf color, adaxial surface — Green, nearest to RHS NN137A. Mature leaf color, abaxial surface — Yellow-green to greyed-green, nearest to a blend of RHS 147B and 191A. Venation — Type — Pinnate. Vein color, adaxial surface — Yellow-green, nearest to a blend of RHS 144A and 144B. Vein color, abaxial surface — Yellow-green, nearest to RHS 146D.
   Petiole.—Length — 18.8 cm. Diameter — 0.3 cm. Strength — Strong. Texture, adaxial and abaxial surfaces — Sparsely covered with thin strigose hairs with an average length of 0.1 cm; hairs are a translucent white, nearest to RHS NN155D. Luster, adaxial and abaxial surfaces — Glossy. Color, adaxial surface — Yellow-green, nearest to RHS N144C. Color, abaxial surface — Yellow-green, nearest to RHS N144C.
   Stipules: General — Two present at the base of each leaf. Shape — Lanceolate. Length — Average of 2.5 cm. Width — Average of 0.5 cm. Apex — Narrowly acute. Base — Broad cuneate. Cross-section — Prostrate and convex. Color, adaxial surface — Yellow-green, nearest to a blend of RHS 145A and 145B. Color, abaxial surface — Yellow-green, nearest to a blend of RHS 145A and 145B. Texture, adaxial surface — Glabrous. Texture, abaxial surface — Sparsely covered in pubescence with an average length of 0.1 cm; hairs are a translucent white, nearest to RHS NN155D.
Inflorescence:
   Type.—Compound cymose panicle.
   Natural flowering season.—From late April to early November in Ens, Netherlands.
   Time to flower.—9 months.
   Length.—8.0 cm, not including the peduncle.
   Diameter.—5.2 cm.
   Quantity of open flowers per inflorescence.—4.
   Quantity of buds per inflorescence.—27.
   Quantity of flowers and buds per plant.—40.
   Peduncle.—Length — 15.7 cm. Diameter — 0.3 cm. Arrangement — At an average angle of 80 degrees to horizontal. Strength — Strong. Texture — Sparsely covered with pubescence with an average length of 0.75 cm; hairs are a translucent white, nearest to RHS NN155D. Luster — Slightly glossy. Color — Yellow-green, nearest to RHS 144B.
Flower buds:
   Shape.—Ovate.
   Length.—Average of 1.2 cm.
   Diameter.—Average of 1.1 cm.
   Texture.—Smooth and glabrous with the exception of the immature sepals which are moderately covered with thin, adpressed pubescence hairs with an average length of 0.5 mm; hairs are a translucent white, nearest to RHS NN155D.
   Luster.—Matte.
   Color.—Green, nearest to RHS 138B; transitioning to yellow-green proximally to the base, nearest to RHS N144D.
Flower:
   Form.—Rotate; single.
   Attitude.—Upright and outward.
   Flower diameter.—2.3 cm.
   Flower depth.—1.2 cm.
   Fragrance.—Moderately strong, sweet fragrance.
   Flowering habit.—Moderately free-flowering.
   Lastingness.—Approximately 7 days.
   Persistent.—Self-cleaning.
   Pedicels.—Length — 3.0 cm. Diameter — 0.175 cm. Cross-section — Carinate. Attitude — Approximately 35 degrees to the peduncle axis. Strength — Moderately strong. Texture — Moderately covered with strigose pubescence with an average length of 0.5 mm; hairs are a translucent white, nearest to RHS NN155D. Luster — Slightly glossy. Color — Yellow-green, nearest to RHS 144C.
   Petals.—Arrangement — Rotate with a single whorl of free petals. Quantity of Petals — 5 and occasionally 6. Shape — Near orbicular. Length — 1.1 cm. Width — 1.1 cm. Apex — Obtuse. Base — Broad cuneate. Margin — Entire; slightly undulated. Cross-section — Very slightly concave. Texture and luster of upper surface — Glabrous; moderately velvety; matte. Texture and luster of lower surface — Glabrous; moderately velvety; matte. Petal color when opening, upper surface — White, nearest to RHS NN155D. Petal color when opening, lower surface — White, nearest to RHS NN155D. Petal color when fully opened, upper surface — White, nearest to RHS NN155B. Petals do not fade with age. Petal color when fully opened, lower surface — White, nearest to RHS NN155B. Petals do not fade with age. Petal venation color, fully opened, upper surface — Veins are not visible. Petal venation color, fully opened, lower surface — Veins are not visible.
   Calyx.—Shape — Rotate. Length — 0.6 cm. Diameter — 2.4 cm. Sepals — Arrangement — Rotate with sepals in a single whorl and fused at the base. Quantity — 12, on average. Length — 1.0 cm. Width — 0.5 cm. Shape — Elliptic to oblong. Apex — Varying from acute to bifid with acute lobe apices. Base — Fused, cuneate. Sepal lobe margin — Entire; not undulated. Texture, upper surface — Sparsely covered with pubescence with an average length of 0.5 mm; hairs are a translucent white, nearest to nearest to RHS NN155D. Texture, lower surface — Sparsely covered with pubescence with an average length of 0.625 mm; hairs are a translucent white, nearest to nearest to RHS NN155D. Luster, adaxial surface — Matte. Luster, abaxial surface — Matte. Color when opening, upper surface — Green, nearest to RHS 143A. Color when opening, lower surface — Green, nearest to RHS 138B. Color when fully opened, upper surface — Green, nearest RHS 137A. Color when fully opened, lower surface — Green, nearest to RHS 138B.
   Bracts.—Position — Two bracts are present at the base of each pedicel. Shape — Elliptic to obovate. Cross-section — Carinate. Length — 3.3 cm. Width — 2.2 cm. Apex — Acute. Base — Attenuate. Margins — Serrate to dentate-serrate. Color, adaxial surface — Green, nearest to RHS 137A. Color, abaxial surface — Green, nearest to RHS 138B. Texture, adaxial surface — Sparsely covered with pubescence with an average length of 0.5 mm; colored a translucent white, nearest to nearest to RHS NN155D. Texture, abaxial surface — Moderately covered with pubescence with an average length of 0.75 mm; colored a translucent white, nearest to nearest to RHS NN155D.
Reproductive organs:
   Stamens.—Quantity — Approximately 22. Anthers — Attachment — Basifixed. Shape — Broad elliptic to oblong. Length — 0.1 cm. Width — 0.75 mm. Color — Yellow — orange, nearest to RHS 22B. Filaments — Length — Approximately 0.2 cm. Color — Yellow-green, nearest to RHS 145D. Pollen, abundance — Sparse. Pollen, color — Yellow, nearest to in between RHS 13B and 13C.
   Pistil.—Quantity — 300. Length — 0.1 cm. Style — Length — 0.7 mm. Color — Yellow-green, nearest to RHS 151B. Stigma — Shape — Club-shaped. Length — 0.3 mm. Diameter — 0.3 mm. Color — Yellow-green, nearest to RHS 151D.
   Ovary Color.—Yellow-green, nearest to RHS 145B.
Seed and fruit:
   Fruit.—Harvest Season — July through September in the Netherlands. Time to maturity — Approximately 5 weeks after flowering. Quantity — An average of 11 per plant. Dimensions — 3.6 cm long and 3.4 cm in diameter. Shape — Conical to short wedge. Texture, adaxial and abaxial surfaces — Moderately covered with very short, strigose hairs with an average length of 0.05 mm; hairs are a translucent greyed-white, nearest to RHS 156D. Luster — Glossy. Color — Immature fruit — Yellow-green, nearest to RHS 145D yet lighter. Immature flesh — Green-white, nearest to RHS 157D. Mature fruit — Red, nearest to RHS 46A. Mature flesh-Red, nearest to RHS 46C. Fruit flavor — Pleasantly and moderately sweet, non-acidic taste. Firmness of flesh — Moderately firm. Lastingness of fruit — Approximately 1 week. Market use — Fresh fruit market.
   Achenes.—Position — Below the fruit surface. Quantity of achenes per fruit — Approximately 200. Shape — Flattened; outline is ovate. Length — 0.125 cm. Diameter — 0.08 cm. Texture — Smooth; glabrous. Color — Greyed-orange, nearest to RHS 165B.
Comparison with the parents:

Plants of the new cultivar ‘Finette’ may be distinguished from its seed parent, Fragaria x ananassa ‘13-077’ (not patented), by the characteristics described in Table 1.

TABLE 1

| Characteristic | ‘Finette’ | ‘13-077’ |
|---|---|---|
| Foliage abundance. | More abundant than | Less abundant than |
| | ‘13-077’ | ‘Finette’. |
| Fruit taste. | Sweet with no acidic | A pleasant blend of sweet |
| | flavor. | and sour, with sourness |
| | | being the more dominant |
| | | flavor. |

Plants of the new cultivar ‘Finette’ may be distinguished from its pollen parent, Fragaria x ananassa ‘07-075’ (not patented), by the characteristics described in Table 2.

TABLE 2

| Characteristic | ‘Finette’ | ‘07-075’ |
|---|---|---|
| Foliage abundance. | More abundant than | Less abundant than |
| | ‘07-075’. | ‘Finette’. |
| Fruit taste. | Sweet with no acidic | A pleasant sweetness |
| | flavor. | withl ittle to no |
| | | acidic flavor. |

Comparison with the closest known commercial comparator:

Plants of the new cultivar ‘Finette’ may be distinguished from the most similar known commercial comparator, Fragaria x ananassa ‘Gorella’ (not patented), by the characteristics described in Table 3.

TABLE 3

| Characteristic | ‘Finette’ | ‘Gorella’ |
|---|---|---|
| Height. | 27 cm, on average. | 25 cm, on average. |
| The age at which | Nine months. | Twelve months. |
| flowers bloom. | | |
| Fruit color. | Red. | Orange-red. |

That which is claimed is:

1. A new and distinct cultivar of Fragaria x ananassa plant named ‘Finette’, substantially as described and illustrated herein.
